(19) United States
(12) Patent Application Publication
(10) Pub. No.: US 2026/0294566 A1
(43) Pub. Date: Oct. 1, 2026

(54) METHOD FOR KEEPING A SURGICAL INSTRUMENT OF A ROBOTIC SURGERY SYSTEM, DURING ITS MOVEMENT CONTROL, WITHIN THE FIELD OF VIEW OF A VIEWING SYSTEM AND RELATED ROBOTIC SURGERY SYSTEM

(71) Applicant: MEDICAL MICROINSTRUMENTS, INC., Wilmington, DE (US)

(72) Inventors: Emanuele RUFFALDI, Pisa (IT); Massimiliano SIMI, Pisa (IT)

(21) Appl. No.: 19/478,707

(22) PCT Filed: Apr. 22, 2024
(86) PCT No.: PCT/IB2024/053901

§ 371 Date: Oct. 24, 2025

(30) Foreign Application Priority Data
Apr. 26, 2023 (IT) 102023000008145

(51) Int. Cl. A61B 34/35; A61B 34/20; A61B 90/00; B25J 13/02; G06T 7/70; G06T 11/60; G16H 40/67; H04N 23/667; H04N 23/69

(52) U.S. Cl. CPC A61B 34/35; A61B 90/361; B25J 13/02; G06T 7/70; G06T 11/60; G16H 40/67; H04N 23/667; H04N 23/69; A61B 2034/2065; A61B 2090/371; A61B 2090/373; G06T 2207/10016; G06T 2207/30004; G06T 2210/41

(57) ABSTRACT

A method controls a robotic system for medical or surgical teleoperation. The robotic system includes a surgical instrument, for teleoperation, and a viewer displaying images and/or videos of a viewing space associated with a teleoperation area in which the surgical instrument operates. It is determined whether a position of the surgical instrument is within an allowed space correlated to a first viewing space, and a current visualization is provided, the position of the surgical instrument is not within the allowed space, the method automatically provides a second viewing space having a surface or field of view greater than and at least partially containing the first viewing space. The second visualization includes a combination and/or superimposition of the first and second viewing spaces, and/or a switching from the first to second viewing space. Switching is performed by controlling optical and/or digital parameters. A robotic system is controlled by the method.

### CROSS-REFERENCE TO RELATED APPLICATIONS

This application is a National Stage Application of PCT/IB2024/053901, filed Apr. 22, 2024, which claims benefit of priority to IT patent application No. 102023000008145 filed on Apr. 26, 2023, and which applications are incorporated herein by reference. To the extent appropriate, a claim of priority is made to each of the above disclosed applications.

### FIELD OF APPLICATION

The present invention relates to a method and system for controlling a robotic system for medical or surgical teleoperation.

In particular, the invention relates to a method (and a related robotic surgery system) for keeping a surgical instrument, teleoperated and controlled in movement, within the field of view of a viewing system with which the robotic surgery system is provided.

### DESCRIPTION OF THE PRIOR ART

In a system for robotic surgery, the field of view (FOV) provided by any viewing system associated therewith (e.g., microscope, exoscope, endoscope) is typically included in the workspace of the slave device (also defined a “slave workspace”).

In other words, often, due to a high use of magnification, or a position of the camera which is very close to the work area, or a small workspace, or simply due to a large workspace of the slave device, the field of view FOV is a subspace, i.e., it represents a subset, of the workspace of the joints of the slave device.

Therefore, a movement of an instrument controlled by the master device can be mapped inside the slave workspace (i.e., inside the space of the slave joints) but outside the effective field of view FOV and therefore is not carried out under the complete control of the operator who, in a robotic teleoperation system, closes the control loop of each movement through his own sight mediated by the viewing system.

For example, executions of surgical gestures such as pulling a suture filament during the passage of the needle in tissues or making a knot, as well as the retraction of an organ or tissue, or a gripping motion of an object at the limits of the field of view FOV, can lead to movements outside the field of view FOV of one or more surgical instruments of the slave device (or, in particular, of articulated terminals, also called “end-effectors”, of such surgical instruments).

It is therefore possible, and in some cases frequent, that for convenience and speed, the user carries and moves an instrument outside the field of view.

However, if not carried out with caution and experience, moving a surgical instrument outside of the field of view can be dangerous and potentially cause damage to the patient such as perforations and/or lacerations of the tissues. This is because robotic tools are usually much stiffer, stronger, or sharper than the tissue can withstand.

From such a situation, a further risk can arise deriving from the subsequent attempt of the operator to re-enter the field of view with the surgical instruments during teleoperation, and moving the instrument “blindly” (when it is no longer in the field of view), thus significantly increasing the risk of damage to the patient; or to attempt an entry into teleoperation through an alignment phase involving movement of the articulated instruments outside of the field of view.

A first solution for overcoming the above drawbacks is to control the movements of the surgical instruments so as to prevent them from exiting the field of view FOV, for example by locking the instruments or constraining them in motion so that they remain within the field of view (even when the command of the master device, in the absence of such a safety control, would determine the exit of the slave device, and therefore of the surgical instrument, from the field of view).

Such a solution responds to the requirement of safety, but at the cost of placing very strict constraints on usability, or ease of use, thus generating inconveniences from this point of view.

In light of this, the need remains to combine the requirement of safety (i.e., avoiding the risk of causing damage to the patient when the surgical instrument is not visible) and the requirement of easy usability, reducing, as much as possible, the constraints imposed by rigidly confining and in any case the movement of the surgical instrument within a field of view originally defined by the viewing system.

### SUMMARY OF THE INVENTION

It is an object of the present invention to provide a method for controlling a slave device, controlled by a master device in a robotic system for medical or surgical teleoperation, taking into account limits of a field of view, which allows at least partially obviating the drawbacks complained above with reference to the prior art, and responding to the aforementioned needs particularly felt in the technical field considered.

### BRIEF DESCRIPTION OF THE DRAWINGS

Further features and advantages of the method according to the invention will become apparent from the following description of preferred embodiments, given by way of non-limiting indication, with reference to the accompanying drawings, in which:

FIGS. 1A, 1B, 2A and 2B show a robotic system, in accordance with the invention, according to some possible embodiments;

FIG. 2C diagrammatically shows a detail of a robotic system, according to an embodiment of the present invention;

FIG. 3 diagrammatically shows an image acquisition device with a viewing space including a surgical site and a slave surgical instrument, according to an embodiment of the present invention;

FIGS. 4, 5, 6 and 7 diagrammatically show some possible configurations of a viewing space of an image acquisition device, according to some embodiments of the present invention;

FIGS. 8A, 8B and 8C are block diagrams schematically showing a robotic system, according to some embodiments of the present invention;

FIGS. 9, 10A, 10B and 11 are block diagrams showing some possible steps of a control method, according to some embodiments of the present invention;

FIGS. 12A, 12B, 12C, 12D, 13 and 14 are views of a screen displaying images of two viewing spaces, according to some possible embodiments of the present invention;

FIGS. 15A, 15B, 16A and 16B are diagrammatic views of an image acquisition device, according to some embodiments of the present invention;

FIGS. 17A and 17B diagrammatically show some possible configurations of a viewing space, according to some embodiments of the present invention;

FIGS. 18A, 18B and 18C diagrammatically show a sequence of some possible steps of a control method, according to an embodiment of the present invention;

FIGS. 19A and 19B diagrammatically show a sequence of some possible steps of a control method, according to an embodiment of the present invention;

FIGS. 20A, 20B and 20C diagrammatically show a sequence of some steps of a control method, according to an embodiment of the invention;

FIGS. 21A-21D diagrammatically show a sequence of some steps of a control method, according to an embodiment of the present invention;

FIG. 21E is a diagram of some possible steps of a control method, according to an embodiment of the present invention;

FIGS. 22A, 22B, 22C and 23 show display modes included in some embodiments of the method according to the invention.

### DETAILED DESCRIPTION

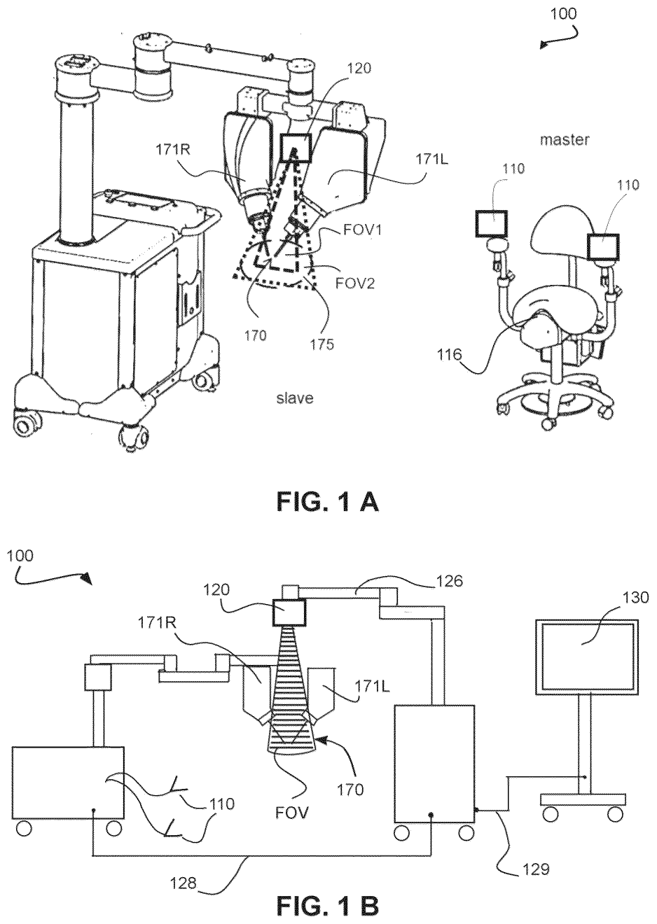

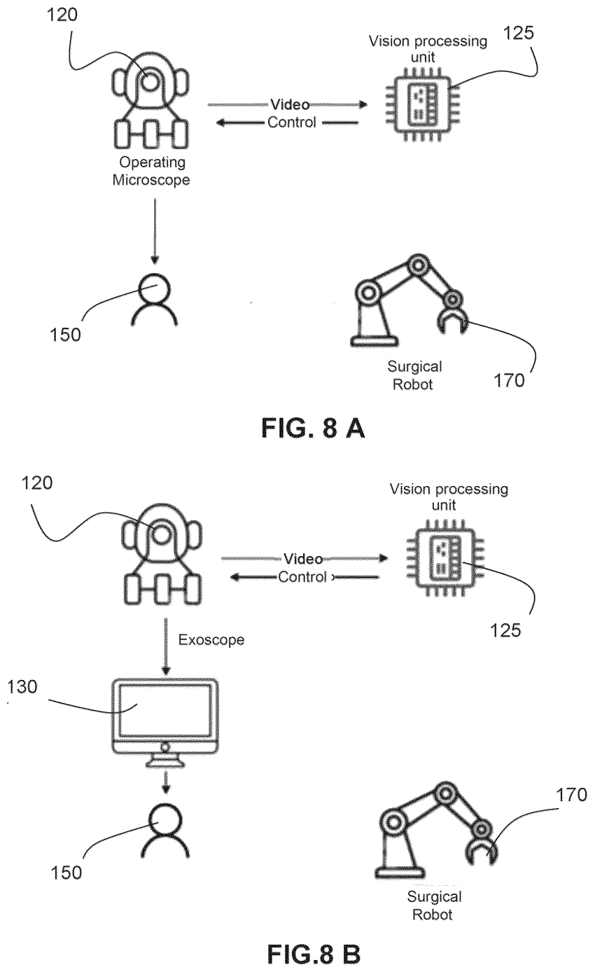

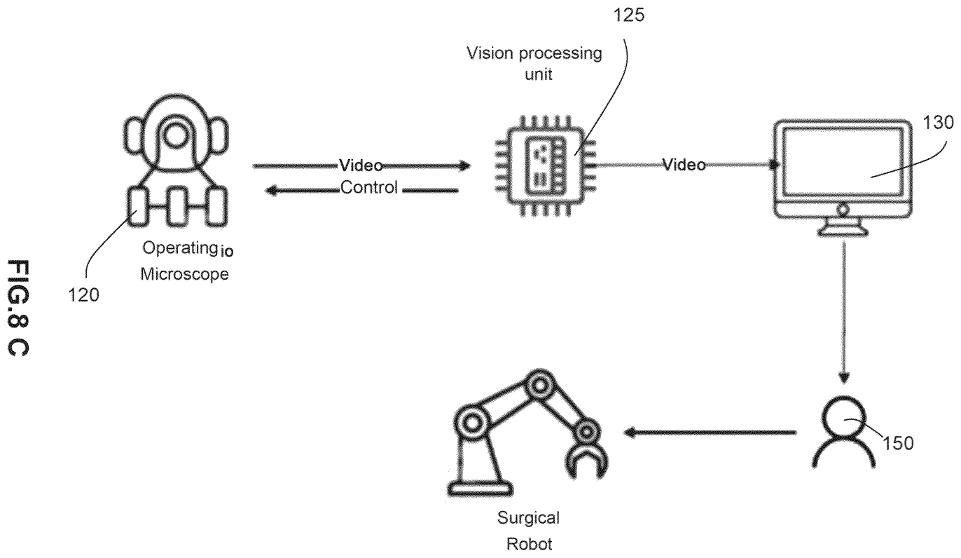

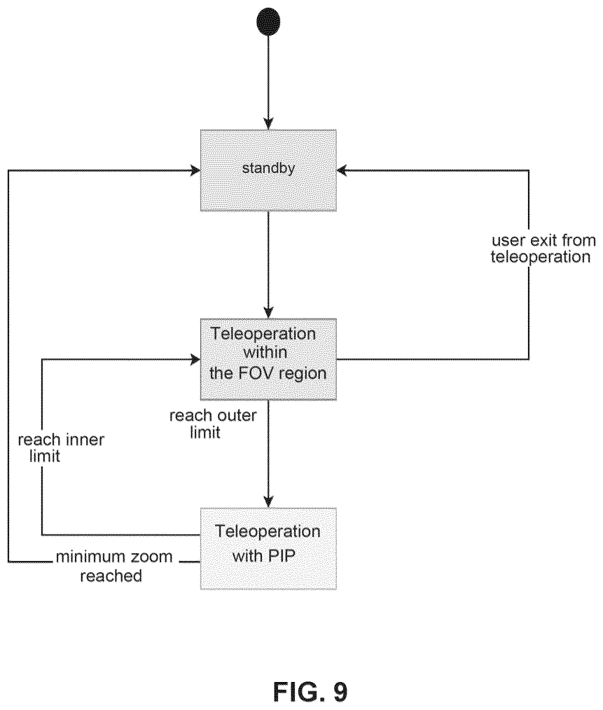

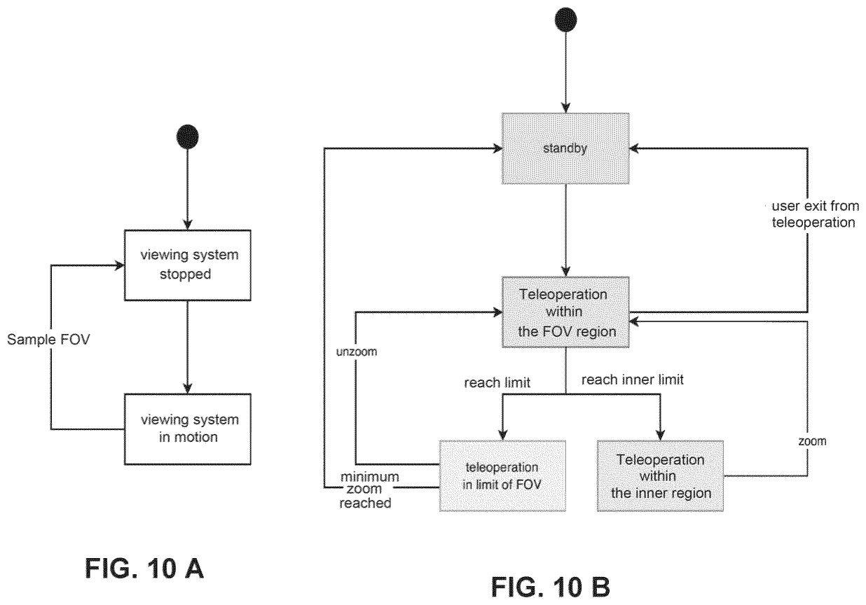

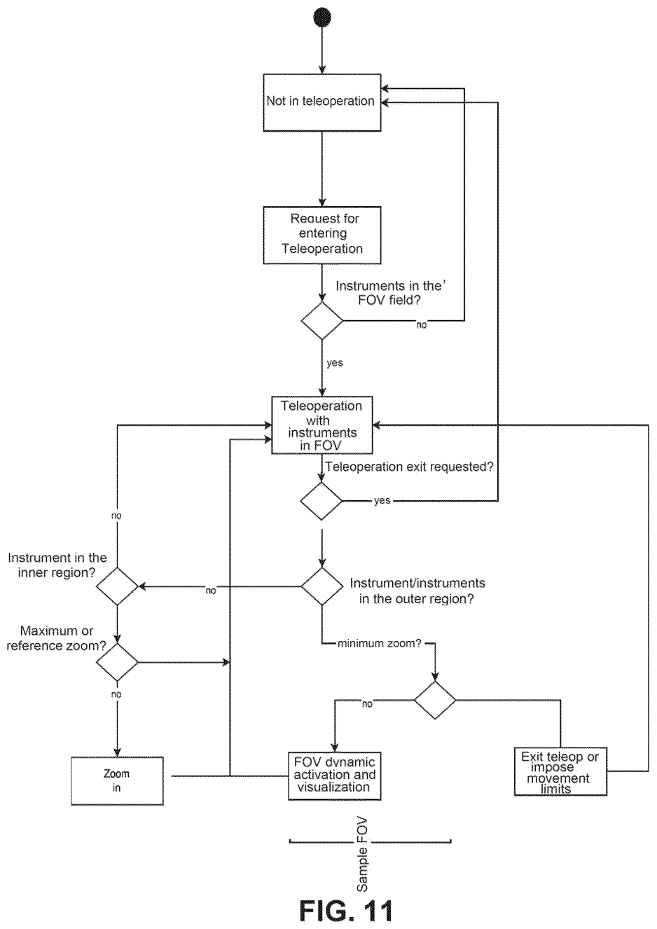

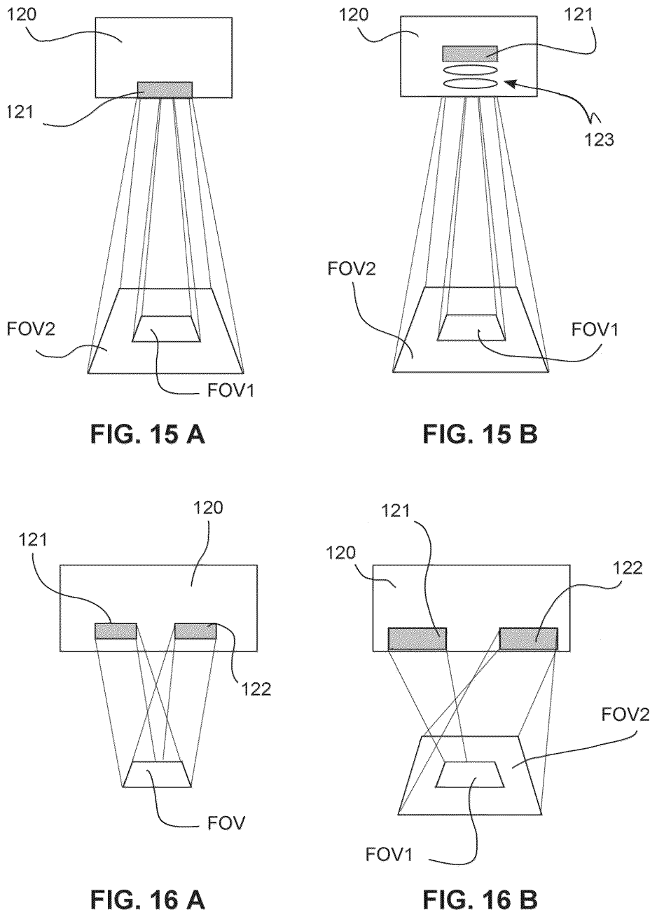

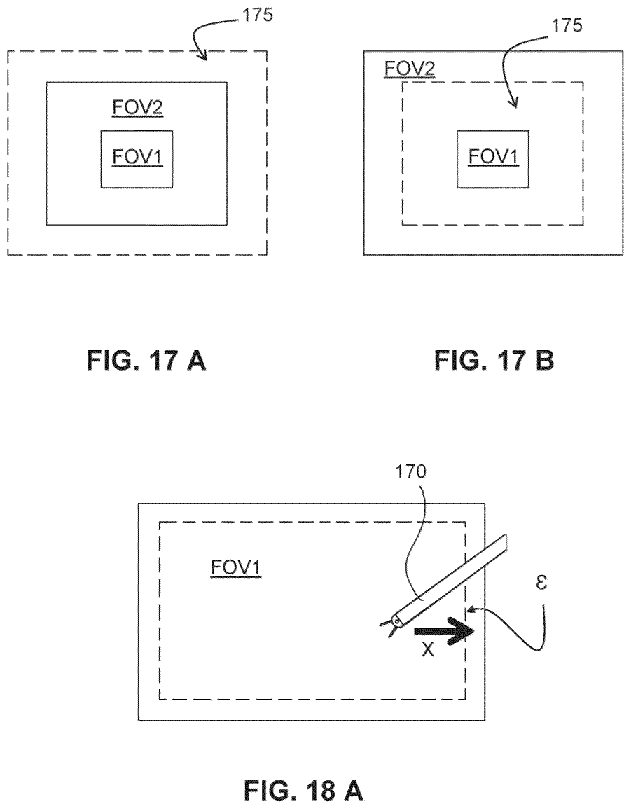

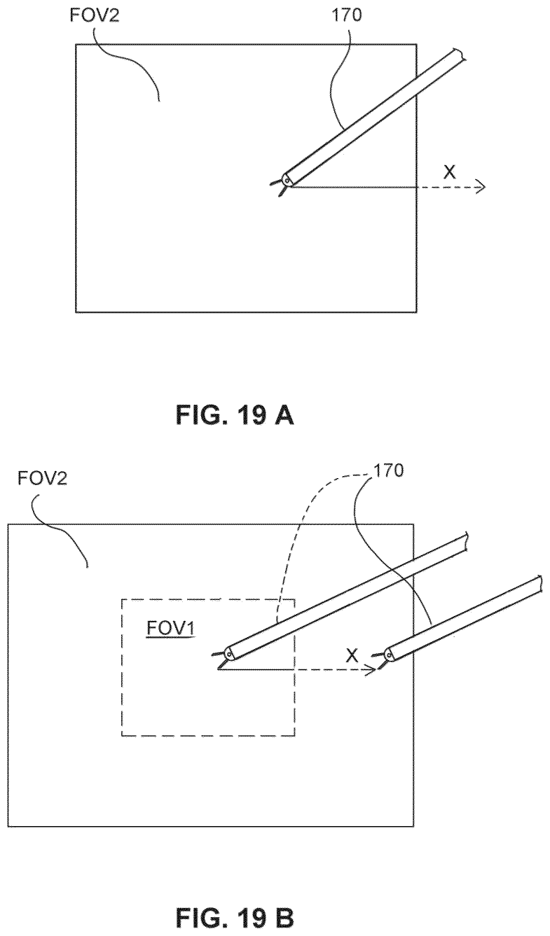

With reference to FIGS. 1-23, a method for controlling a robotic system for medical or surgical teleoperation is described.

The robotic system, to which the method is applied, comprises at least one surgical instrument 170, adapted to operate in teleoperation, and further comprises viewing means 120, 130 configured to display to an operator 150 images and/or videos of at least one viewing space associated with a teleoperation area in which the surgical instrument 170 operates.

The method first comprises the step of determining whether a position of the surgical instrument 170 is within an allowed space correlated to a first viewing space FOV1, in which the first viewing space FOV1 is used as the current viewing space for the teleoperation, with respect to which a current visualization is provided to the operator.

The method then comprises, if or when it is determined that the aforesaid position of the surgical instrument 170 is not within the aforesaid allowed space correlated to the first viewing space FOV1, the further step of automatically providing, by the viewing means, a second visualization defining a second viewing space FOV2 having a surface or field of view greater than the aforesaid first viewing space FOV1 and containing, or partially containing, the aforesaid first viewing space FOV1.

Said second visualization comprises a combination and/or superimposition of the aforesaid second viewing space FOV2 and first viewing space FOV1, and/or a switching from the aforesaid first viewing space FOV1 to the aforesaid second viewing space FOV2, in which such a switching is performed, without mechanical movements, by controlling optical and/or digital parameters of the viewing means 120, 130.

According to an embodiment of the method, the aforesaid step of automatically providing a second visualization comprises displaying to the operator 150 a combination and/or superimposition of the aforesaid second viewing space FOV2 and of the aforesaid first viewing space FOV1, for example in images and/or videos, by display means 130 included in the aforesaid viewing means.

In accordance with an embodiment of the method, the aforesaid step of automatically providing a second visualization comprises displaying to the operator 150 the second viewing space FOV2, through a switching from the aforesaid first viewing space FOV1 to the aforesaid second viewing space FOV2, by controlling optical and/or digital parameters of image acquisition means 120 comprised in the aforesaid viewing means, in which such control and such switching are performed without carrying out mechanical movements.

According to possible implementation options, the aforesaid optical parameters comprise, for example, electronic control of zoom and/or exposure and/or focus and/or depth of focus, and the aforesaid digital parameters comprise, for example, digital scale factor and/or magnification and/or region of interest and/or digital zoom or other digital image processing.

According to an embodiment of the method, the aforesaid step of determining a position of the surgical instrument 170 comprises determining a current position of the surgical instrument 170 with respect to the allowed space correlated to the first viewing space FOV1, and/or the presence of the surgical instrument 170 in the allowed space correlated to the viewing space.

In such a case, the aforesaid step of automatically providing a second visualization comprises automatically providing a second visualization when the current determined position of the surgical instrument 170 is on the boundary and/or outside the allowed space correlated to the first viewing space FOV1.

According to an embodiment of the method, the aforesaid current portion of the surgical instrument 170 is detected based on digital data, for example digital images, provided in real time by image acquisition means 130 included in the viewing means.

In accordance with an embodiment, the method is applied in a robotic system comprising at least one master device 110 adapted to be moved by an operator 150, and further comprising the aforesaid at least one surgical instrument 170 adapted to be controlled by the master device 110.

In such a case, said step of determining a position of the surgical instrument 170 comprises determining a position of the surgical instrument 170 with respect to the first viewing space FOV1, as imposed by the master device 110.

The aforesaid step of automatically providing a second visualization comprises automatically providing a second visualization when the controlled position of the surgical instrument 170, as was determined, is on the boundary and/or outside the aforesaid allowed space correlated to the first viewing space FOV1.

According to an embodiment of the method, the aforesaid step of automatically providing a second visualization comprises ensuring that the surgical instrument is displayed in/from the second visualization provided to the operator, during the movement of the surgical instrument 170 as controlled by the master device 110, even when the surgical instrument moves outside the allowed space correlated to the first viewing space FOV1, during teleoperation or preparation for teleoperation.

According to an embodiment of the method, the aforesaid second viewing space FOV2 is a physical viewing space, detectable and displayable by the viewing means in addition to the first viewing space FOV1. Such a second viewing space FOV2 is wider than the first viewing space FOV1 and/or contains the first viewing space FOV1 (i.e., in other words, the first viewing space FOV1 is included in the second viewing space FOV2).

According to another embodiment of the method, the aforesaid second viewing space FOV2 is a virtual viewing space, extractable or extrapolable from digital images and/or videos previously detected and/or displayable by the viewing means in addition to the first viewing space FOV1. Such a second viewing space FOV2 is wider than the first viewing space FOV1 and/or contains the first viewing space FOV1 (i.e., in other words, the first viewing space FOV1 is included in the second viewing space FOV2).

In accordance with an embodiment, the method comprises the further steps of acquiring, by the viewing means, both a first digital video image of a magnified operating scenario, defined in the aforesaid first viewing space FOV1, and a second digital video image of the enlarged operating scenario, defined in the aforesaid second viewing space FOV2, wider and/or at a lower zoom with respect to the first viewing space FOV1, which includes the first viewing space FOV1; the surgical instrument is displayed in the second viewing space FOV2.

In such a case, the aforesaid step of automatically providing a second visualization, when the determined position of the surgical instrument is on the boundary or outside the allowed space correlated to the first viewing space FOV1, comprises displaying both the first viewing space FOV1 and the second viewing space FOV2.

According to an implementation option of such an embodiment, the step of displaying both the first viewing space FOV1 and the second viewing space FOV2 comprises displaying the second viewing space FOV2, in which the surgical instrument is visible, in overlay on a screen portion 130, covering a part of the first viewing space.

According to an implementation option, the screen portion in which the second viewing space FOV2 is in overlay comprises an area located at one of the four corners of the screen 130.

According to another implementation option, the screen portion 130 in which the second viewing space FOV2 is in overlay comprises a side box, arranged on the side of the first viewing space FOV1 from which the surgical instrument exited the space correlated to the first viewing space.

According to another implementation option, the aforesaid step of displaying both the first viewing space FOV1 and the second viewing space FOV2 comprises displaying the second viewing space FOV2, in which the surgical instrument is visible, in full screen, and displaying the first viewing space FOV1 in overlay on a screen portion, covering a part of the second viewing space in which the surgical instrument is not present.

According to an implementation option, the method further comprises highlighting the second viewing space FOV2, by means of increased brightness or with a colored or bright edging, at the moment of transition between the first visualization and the second visualization.

According to an implementation option, the aforesaid step of displaying both the first viewing space FOV1 and the second viewing space FOV2 comprises displaying the first viewing space FOV1 on a first screen and displaying the second viewing space FOV2 on a second screen.

In accordance with another embodiment, the method comprises the further steps of acquiring, by the viewing means, both a first digital video image of a magnified operating scenario, defined in the aforesaid first viewing space FOV1, and a second digital video image of the enlarged operating scenario, defined in the aforesaid second viewing space FOV2 (wider and/or at a lower zoom with respect to the first viewing space FOV1), which includes the first viewing space FOV1; the surgical instrument is displayed in the second viewing space FOV2.

In such a case, the aforesaid step of automatically providing a second visualization, when the determined position of the surgical instrument is on the boundary or outside the allowed space correlated to the first viewing space FOV1, comprises processing both the first acquired digital video image and the second acquired digital video image, by image processing techniques, so as to perform a pixel-by-pixel merging of the two digital images and thus obtain, as a second visualization provided to the operator, a combined digital image of the operating scenario, based on the aforesaid image merging.

The aforesaid combined digital image has a higher resolution and/or a greater field depth with respect to the aforesaid first acquired digital video image and second acquired digital video image.

According to an embodiment of the method, the aforesaid steps of acquiring a first digital video image and a second digital video image are carried out simultaneously, continuously and in real time, by means of two image sensors or cameras, included in the viewing means, having the same viewpoint or two different respective viewpoints.

According to several possible implementations, said two or more sensors comprise two sensors adapted to provide two two-dimensional viewing spaces FOV, or two sensors adapted to provide one three-dimensional viewing space FOV, or four sensors adapted to provide two three-dimensional viewing spaces FOV.

According to an implementation option, the aforesaid two image sensors comprise two cameras having as viewing space the first viewing space FOV1 and the second viewing space FOV2, respectively, aligned with each other and/or coaxial.

In accordance with an embodiment of the method, the aforesaid steps of acquiring a first digital video image and a second digital video image are carried out alternatively, by means of a single image sensor or camera.

According to an implementation option, such a camera detects the first digital video image associated with the first viewing space FOV1 by adjusting the zoom to a first zoom value, and detects the second digital video image associated with the second viewing space FOV2 by adjusting the zoom to a second zoom value.

According to an implementation option, switching from the first digital video image to the second digital image, or vice versa, involves, in addition to switching from the first zoom value to the second zoom value, or vice versa, also an automatic controlled variation and/or automatic adjustment of other optical parameters, inter alia exposure/brightness and/or focus.

In accordance with another embodiment of the method, the step of providing the second visualization comprises switching from a first digital video image of the operating scenario, taken at a first zoom value, to a second digital image of the operating scenario characterized by a second zoom value less than the first zoom value, and thus from a second viewing space FOV2 which is wider than the first viewing space FOV1, in which the surgical instrument is displayed.

In such a case, the aforesaid switching step is carried out by the viewing means by controlling optical and/or digital parameters, and not by controlling movement parameters of the viewing means.

According to an embodiment, the method includes providing and using a plurality of larger second viewing spaces FOV2₁-FOV2ₙ, characterized by a respective plurality of gradually lower second zoom values.

In such an embodiment, the aforesaid step of automatically providing a second visualization, when the determined position of the surgical instrument is on the boundary of or outside an allowed space correlated to a second current viewing space FOV2ᵢ, comprises displaying the next wider second viewing space FOV2ᵢ₊₁, having a respective lower second zoom value, and such as to display the surgical instrument.

According to an implementation option of the method, if the surgical instrument returns within the allowed space associated with the first viewing space FOV1, the first visualization is restored.

In accordance with an embodiment, the method envisages that the robotic system remains in teleoperation until the surgical instrument is displayed within the aforesaid second visualization.

In accordance with another embodiment, the method provides for the robotic system exiting the teleoperation state, if the surgical instrument reaches the boundaries of the second field of view FOV2, and the second field of view FOV2 cannot be further enlarged.

In accordance with another embodiment, the method provides for the robotic system remaining in a limited teleoperation state, if the surgical instrument reaches the boundaries of the second field of view FOV2, and the second field of view FOV2 cannot be further enlarged.

In the aforesaid limited teleoperation state, the movement of the surgical instrument is only allowed if the surgical instrument is located within an allowed space correlated to the second viewing space and the movement of the surgical instrument, if allowed, is still confined within such a second viewing space.

According to an embodiment of the method, the aforesaid allowed space correlated to the viewing space corresponds to the viewing space.

According to another embodiment of the method, the aforesaid allowed space correlated to the viewing space comprises a subset of the viewing space, corresponding to the viewing space from which an internal surrounding extending with a spatial tolerance ϵ inside the boundaries of the viewing space is removed.

According to an implementation option of the method, the aforesaid viewing space is defined by a field of view (FOV) of the viewing means.

Such an implementation option refers to a robotic system having viewing means, or a generic viewing system (comprising digital image/video acquisition means), capable of capturing a portion of the world observed through appropriate lens or light guide systems.

With a terminology known in the technical field considered, such a portion of the world of which an image or video is acquired has an extension referred to as the “Field of View” (FOV) which is typically represented in angular units taken along the diagonal or one of the axes of the digital image/video acquisition system.

According to another implementation option of the method, said viewing space is defined by a predefined subset of the field of view (FOV) of the viewing means.

In fact, the boundaries of the area or volume defining the viewing space of interest do not necessarily coincide with the field of view; such boundaries can be constructed on a sub-volume of the field of view where the view is optimal and/or in such a way to have a particular geometric shape and/or specially chosen to promote the mobility of the slave device therein and/or for any other reason.

According to another implementation option of the method, the aforesaid viewing space is defined by a field-of-view workspace, consisting of a geometric volume, in a reference coordinate system of the robotic system, associated with the aforesaid field of view.

Such a field-of-view workspace (hereinafter also referred to as “FOV Workspace”) can for example correspond to a volume, e.g., a trapezoid which goes from the lens to infinity and centered in the main axis of the optical system, which is capable of representing the field of view of a digital viewing system, for example for lenses with “Fields of View” FOV less than 180 degrees. Once a plane is fixed with respect to the lens, it is possible to evaluate the extension of the field of view in metric terms by evaluating the portion of the plane which intersects the “FOV Workspace”, and generally such a plane is orthogonal to the main axis.

According to another implementation option of the method, the aforesaid viewing space is defined by geometric limits of the field of view, consisting of a boundary surface of the aforesaid viewing workspace, in the reference coordinate system of the robotic system.

For example, the field-of-view workspace is constructed with respect to the trapezoid originating in the camera image plane of the viewing system. It is possible to build simplified geometries called “field of view workspace limits” (FOV Workspace Limits) therefrom which are imposed to limit the movement of the slave device. Such geometries can be defined as planes orthogonal to the viewing system or as curved surfaces which in any case are defined inside the field-of-view workspace.

According to several possible embodiments, the aforesaid viewing means comprise at least one camera 120 or comprise an endoscope and/or a laparoscope and/or a microscope and/or an exoscope.

According to an implementation option, the viewing means comprise a stereoscopic viewing system comprising two cameras, each of which defines a respective “FOV Workspace” 175, for example right “FOV Workspace” and left “FOV Workspace”. The intersection of the aforesaid two field-of-view workspaces of right and left camera produces a “common field-of-view workspace” which ensures the maximum visibility of the objects in the scene.

In accordance with an embodiment of the method, already mentioned above, the aforesaid step of determining a position of the surgical instrument 170 with respect to the allowed space correlated to the viewing space comprises determining a current position of the surgical instrument 170 and/or the presence of the surgical instrument 170 in the allowed space correlated to the viewing space, based on digital data deriving from the viewing means.

According to another embodiment of the method, the aforesaid step of determining a position of the surgical instrument 170 with respect to the allowed space correlated to the viewing space comprises:
mapping the aforesaid allowed space correlated to the viewing space in a corresponding slave field-of-view workspace, in a slave reference coordinate system associated with the slave device;
determining the current position of the surgical instrument 170 in terms of respective position coordinates in the aforesaid slave reference coordinate system;
determining the current position of the surgical instrument 170 with respect to the allowed space correlated to the viewing space based on a comparison between the aforesaid position coordinates and the aforesaid slave field-of-view workspace, in the slave reference coordinate system.

According to an embodiment of the method, the aforesaid step of determining a position of the surgical instrument 170 with respect to the allowed space correlated to the viewing space comprises calculating and/or determining the position of a real point belonging to the surgical instrument or the position of a virtual point integral with the surgical instrument 170, based on images provided by said viewing system.

According to an implementation option, the aforesaid step of determining a position of the surgical instrument 170 comprises determining the position of a virtual control point 600 of the slave device (for example placed between the tips 171, 172 or jaws 171, 172 of the surgical instrument 170).

According to another implementation option, the aforesaid step of determining a position of the surgical instrument 170 comprises determining the position of at least one of the tips 171, 172 of the surgical instrument 170.

According to an implementation, the aforesaid step of determining a position of the surgical instrument 170 comprises determining the position of at least one of the links of a hinged wrist (or “end-effector”) 177 included in the surgical instrument 170.

According to another implementation option, the aforesaid step of determining a position of the surgical instrument 170 comprises determining the position of a distal portion of a positioning shaft near the hinged wrist of the surgical instrument 170.

In accordance with an embodiment, the method comprises the further step of dynamically adjusting/varying the viewing space, by controlling the viewing means, e.g., by changing the zoom or adjusting the point of view, so as to improve or restore the view of the surgical instrument through the viewing means.

In particular, in an embodiment, the robotic system coupled to the viewing system is capable of acting autonomously on the zoom by widening the viewing space (e.g., FOV) when an instrument reaches the limits of the field of view, thereby preventing a maneuver of the surgeon, possibly involuntary, from causing the exit of the instrument from the field of view.

In an embodiment, a first zoom value related to a first viewing space (e.g., first FOV) and a second zoom value related to a second viewing space (e.g., second FOV) are stored, wherein:
the aforesaid first zoom value is greater than the second zoom value;
the aforesaid first viewing space (e.g., first FOV) is smaller than the aforesaid second viewing space (e.g., second FOV).

The self-adjustment of the zoom upon reaching the limits imposed by the viewing space (e.g., FOV) can vary between the aforesaid first zoom value and second zoom value and the related first FOV and second FOV.

For example, said varying zoom self-adjustment is an intermediate variation between the two values calculated and evaluated based on the target position of the instrument or upon reaching the limits, generating intermediate viewing spaces contained between the first and second viewing spaces, or is one of the two zoom values, and passes from one to the other when the instrument is outside or inside said first viewing space (e.g., FOV).

The switching between the first zoom value and the second zoom value or vice versa does not include any mechanical movement of joints, lenses or microscope but only digital processing.

Still referring to FIGS. 1-23, a robotic system 100 for medical or surgical teleoperation, provided in the present invention, is described below.

Such a robotic system comprises at least one surgical instrument 170, adapted to operate in teleoperation; viewing means 120, 130 configured to display to the operator 150 images and/or videos of at least one viewing space associated with a teleoperation area in which the surgical instrument 170 operates; and lastly a control unit configured to control the surgical instrument 170.

The control unit is further configured to carry out the following actions:
determining whether a position of the surgical instrument 170 is within an allowed space correlated to a first viewing space FOV1, in which such a first viewing space FOV1 is used as the current viewing space for the teleoperation, with respect to which a current visualization is provided to the operator;
automatically providing a second visualization, by the viewing means 120, 130, when in the aforesaid determining step it is determined that the position of the surgical instrument 170 is not within the allowed space correlated to the first viewing space FOV1.

The aforesaid second visualization defines a second viewing space FOV2 having a greater surface or a greater field of view than the aforesaid first viewing space FOV1 and containing, or partially containing, the first viewing space FOV1.

Furthermore, the aforesaid second visualization comprises a combination and/or superimposition of the aforesaid second viewing space FOV2 and first viewing space FOV1, and/or a switching from the first viewing space FOV1 to the second viewing space FOV2, in which such a switching is performed, without including mechanical movements, by controlling optical and/or digital parameters of the viewing means 120, 130.

According to an embodiment of the system, the aforesaid second visualization is representative of a combination and/or superimposition of the second viewing space FOV2 and the first viewing space FOV1.

According to an embodiment, the robotic system further comprises at least one master device 110, adapted to be moved by an operator 150, and at least one slave device comprising the aforesaid surgical instrument 170 adapted to be controlled by the master device.

According to an implementation option, the master device 110 is preferably a “groundless”-type master device, without force feedback, for mono-lateral teleoperation. For example, therefore, the master device can be a master mechanically constrained to an operating console and at the same time be of the “groundless”-type without force feedback, for mono-lateral teleoperation.

According to an implementation option, the master device 110 is preferably a master device of a type which is mechanically unconstrained to the operating console.

In accordance with an embodiment of the system, the viewing means 120, 130 comprise image acquisition means 120, configured to acquire a digital image or video associated with the aforesaid first viewing space FOV1 and/or second viewing space FOV2; and further comprise display means 130, configured to display to the operator 150 the first viewing space FOV1 or the second viewing space FOV2, and/or a combination and/or superimposition of the aforesaid second viewing space FOV2 and first viewing space FOV1.

According to possible implementation options, the viewing means comprise at least one image acquisition device 120, such as at least one camera 120 and/or at least one endoscope, and a screen 130 or display 130, in operating communication therebetween, for example by means of the provision of a vision processing unit.

In accordance with an embodiment of the robotic system, the viewing means are further configured to acquire both a first digital video image of a magnified operating scenario, defined in the first viewing space FOV1, and a second digital video image of the enlarged operating scenario, defined in the second viewing space FOV2, wider and/or at a lower zoom with respect to the first viewing space FOV1, which includes the first viewing space FOV1; the surgical instrument is displayed in such a second viewing space FOV2.

In such a case, the aforesaid action of automatically providing a second visualization, when the determined position of the surgical instrument is on the boundary or outside the allowed space correlated to the first viewing space FOV1, comprises displaying both the first viewing space FOV1 and the second viewing space FOV2.

In accordance with another embodiment of the robotic system, the viewing means are further configured to acquire both a first digital video image of a magnified operating scenario, defined in the first viewing space FOV1, and a second digital video image of the enlarged operating scenario, defined in the second viewing space FOV2, wider and/or at a lower zoom with respect to the first viewing space FOV1, which includes the first viewing space FOV1 and in which the surgical instrument is displayed.

In such a case, the control means, to perform the aforesaid step of automatically providing a second visualization, when the determined position of the surgical instrument is on the boundary or outside the allowed space correlated to the first viewing space FOV1, are further configured to process both the first acquired digital video image and the second acquired digital video image, by image processing techniques, so as to perform a pixel-by-pixel merging of the two digital images and thus obtain, as a second visualization provided to the operator, a combined digital image of the operating scenario, based on the aforesaid image merging.

According to an embodiment of the robotic system, the image acquisition means 120 comprise two or more image sensors or cameras, having the same or two different respective viewpoints, configured to perform the aforesaid actions of acquiring a first digital video image and a second digital video image simultaneously, continuously and in real time.

According to different implementation options, comprised in the present invention, the aforesaid two or more image sensors or cameras comprise two sensors or cameras adapted to provide two two-dimensional viewing spaces FOV, or two sensors or cameras adapted to provide one three-dimensional viewing space FOV, or four sensors or cameras adapted to provide two three-dimensional viewing spaces FOV.

According to a particular implementation option, the aforesaid two image sensors comprise two cameras having as viewing space the aforesaid first viewing space FOV1 and the aforesaid second viewing space FOV2, respectively, aligned with each other and/or coaxial.

According to an implementation example, the image acquisition means 120 comprise a single image sensor or camera, configured to alternately perform the aforesaid actions of acquiring a first digital video image and a second digital video image.

According to an implementation option, the aforesaid image sensor or camera is configured to detect the first digital video image associated with the first viewing space FOV1 by adjusting the zoom to a first zoom value, and to detect the second digital video image associated with the second viewing space FOV2 by adjusting the zoom to a second zoom value.

In accordance with an implementation option, the aforesaid image sensor or camera is configured to perform, upon switching from the first digital video image to the second digital image, or vice versa, in addition to switching from the first zoom value to the second zoom value, or vice versa, also an automatic controlled variation and/or automatic adjustment of other optical parameters, including exposure/brightness and/or focusing.

According to possible implementation options of the robotic system, the image acquisition means 120 comprise an exoscope or a microscope or an endoscope.

According to possible implementation options of the robotic system, the display means 130 comprise at least one electronic screen or monitor.

According to an embodiment, the robotic system further comprises processing means configured to process the digital images or videos acquired by the image acquisition means in order to detect the presence or absence of the surgical instrument 170 in the allowed space correlated to the first viewing space FOV1.

According to several possible implementation options of the robotic system, the control unit is configured to carry out a method for controlling a robotic system according to any one of the embodiments of the method illustrated in this description.

With reference again to FIGS. 1-23, further details will be provided below, by way non-limiting example, with reference to the operating principles and some particular embodiments of the method and system according to the present invention.

Firstly, to show the geometric aspects of the viewing space (or field of view FOV) in more detail, the definitions of the following parameters or quantities are provided:
“Working Distance” [mm] is the distance of the Focal Plane which is the region most in focus;
“Field of View” [degrees] is the extension of the observable world which can be captured;
“Working Area” [mm] is the diagonal of the area framed by the viewing system at a certain “Working Distance”;
“Depth of Field” [mm] is the extension of the region of focus centered at the “Working Distance”—the greater the depth, the better;
“Zoom” (i.e., “zoom ratio”) is the amount of scaling applied to the “Field of View” obtained through an optical or digital modification;
“Magnification” (i.e., “magnification ratio”) is the ratio between an entity of 1 mm placed at a distance equal to “Working Distance” and the representation thereof on a screen or monitor, and is also proportional to “Zoom”.

The viewing space (or field of view, FOV) can be imagined as the truncated pyramid with the minor plane on the camera lens and the other plane at infinity.

Since the FOV is a portion of space, a containment operator can be defined between two viewing spaces FOV1 and FOV 2, i.e., “FOV2⊃FOV1” which indicates that each point of FOV1 is included in FOV2. The case in which FOV2 has the same origin and main axis as FOV1 is also defined with the indication “FOV2∥⊃FOV1”.

In the example considered here, the “Working Area WA” is a rectangle obtained by the intersection of the FOV with a plane placed at a certain working distance. For each distance from the lens, a “Working Area WA” can be defined, but typically it is intended as the “Working Distance” corresponding to the distance over which the camera focus is placed.

As illustrated in FIG. 3, a “Slave Working Area, SWA” is defined here as the “Working Area WA” defined by the distance of a chosen point of the surgical instrument 170, i.e., the control point thereof along the main axis, for example:

$$SWA = WA ( dS )$$

where dS=distance along the axis Z of the slave device from the camera 120.

In FIG. 3, dF is the focal length of the viewing system, dS is the distance of the instrument from the origin (camera), WA(dF) is the Working Area at dF, WA(dS) is the Working Area at dS, and finally dF (dF) is the field depth at dF.

Such a distinction is appropriate because the operator-controlled surgical instrument will work at a distance dS in the range dV and dF, where dV is for example 100 mm from the exoscopic head, and dF is instead at the center of the surgical site 180 where the focus is placed, for example 250 mm. It is further desirable for the distance dS to be in the region of focus range for the focal length dF: [dF−rF/2, dF+rF/2].

A functional of distance is further defined between the slave device (and the relative surgical instrument) and the limits of the viewing space FOV so that it is positive for points inside the FOV and negative for external points. Such a distance can be expressed either in visual coordinates (pixels), in metric coordinates (mm) or in normalized coordinates.

An example of a distance is the Euclidean one in three-dimensional space between a chosen point of the slave device and the closest point of the contour surface of the FOV.

Another purely planar distance can be obtained by estimating the distance of the slave device from the image edge in pixels or in normalized coordinates.

A “Zoom-Focus” algorithm is illustrated below, according to an implementation example.

A viewing system is given which has two controllable parameters: “Zoom” K, from 1× to Mx, and “Working Distance” F from A to B in millimeters (for example, from 200 mm to 550 mm).

The “Zoom” parameter controls the FOV: the higher the zoom the lower the FOV: for example, FOV(K)=60 deg/K.

The “Working Distance” parameter controls the focusing.

Without losing generality, the “Working Area” can be indicated at a distance d for a Zoom level K equal to: WA(d, K)=2 d tan (FOV(K)/2), where WA(F, K) is the nominal “Working Area” on the focusing plane.

A possible “Working Distance” is not obtainable at each Zoom level and in general it is possible to identify a functional defining such a range for each Zoom level:

$$WD\_range ( K ) = [ A\_K , B\_K ]$$

$$WD\_span ( K ) = B\_K - A\_K$$

The “WD_span” range is maximum for 1× Zoom and decreases for high Zooms.

Similarly, the “Depth Of Field, DoF” parameter is a function of WD: the higher WD, the higher the DoF.

The focus region is a length range DoF(WD) such that the focus region is [WD-DoF (WD)/2, WD+DoF(WD)/2].

The following two problems are then considered:
(i) given an object at a distance d with respect to the lens along the main optical axis, and distance L with respect to the axis itself, find the parameters of K, WD which optimize the point of view;
(ii) given two objects at a distance (d1,L1) and (d2,L2), for example the slave device and the surgical region of interest, look for the parameters K and WD which visibly contain both objects and focus on the two according to a proportional priority p and (1−p). If the focusing of point 1 is favored, then p will be greater than 0.5.

The problem (i) has an ideal solution if it is possible to find K and F:

$$WA ( d , K ) >= 2 L$$

$$d / 2 in [ F - DoF ( F , K ) / 2 , F + DoF ( F , K ) / 2 ]$$

The problem (ii) has an ideal solution if it is possible to find K and F:

$$WA ( d 1 , K 1 ) >= 2 L 1$$

$$WA ( d 2 , K 2 ) >= 2 L 2$$

$$( d 1 p + d 2 ( 1 - p ) ) in [ F - DoF ( F , K ) / 2 , F + DoF ( F , K ) / 2 ]$$

If the problems are not solvable, then the points will not be visible (outside the Field of View) or will not be in Focus (outside of “Focus”).

If the problems are solvable with a range of solutions, then the one which minimizes the change in “Zoom” factor or other visual comfort criteria, or proximity with respect to FOV1, will be preferred.

Now an embodiment is considered comprising a robotic system comprising a master-slave teleoperated robot, at least one associated surgical instrument, at least one master controller, at least one control and calculation unit CPU, and a slave workspace within which the surgical instruments can move.

The teleoperated robot further comprises, or is associated with, an optical or digital viewing system (e.g., microscope, exoscope, or endoscope) defining a viewing space FOV.

The robotic system is connected to the viewing system and is capable of commanding real-time changes of one or more parameters of the viewing system itself (such as zoom level, and/or focal length, and/or brightness, and/or exposure, and/or color filtering, and/or other parameters) based on events or states of the robotic system and/or conditions identified by the overall system consisting of robotic surgical system and viewing system.

According to such an embodiment, the method provides for the aforesaid overall system digitally using and processing the images and data of the viewing system in real time to identify the position or presence or non-presence of one or more surgical instruments (and/or of the respective terminal elements of the slave device, and/or hinged wrist and/or control point) in the viewing space FOV.

The method further provides for, when in a teleoperation state in which at least one surgical instrument (i.e., the related slave device) is in the viewing space FOV, the position commanded to the slave device having a distance from the limits of the viewing space FOV (indicated in the figures as “Outer Limit”) at a threshold value “EPS” or being outside the workspace FOV, then at least one new FOV (second viewing space FOV2) or a representation thereof containing the at least one surgical instrument is provided to the user and/or has always kept it inside the second viewing space FOV2 during the movement thereof by automatically modulating the optical/digital parameters of the viewing system (but without physical movement).

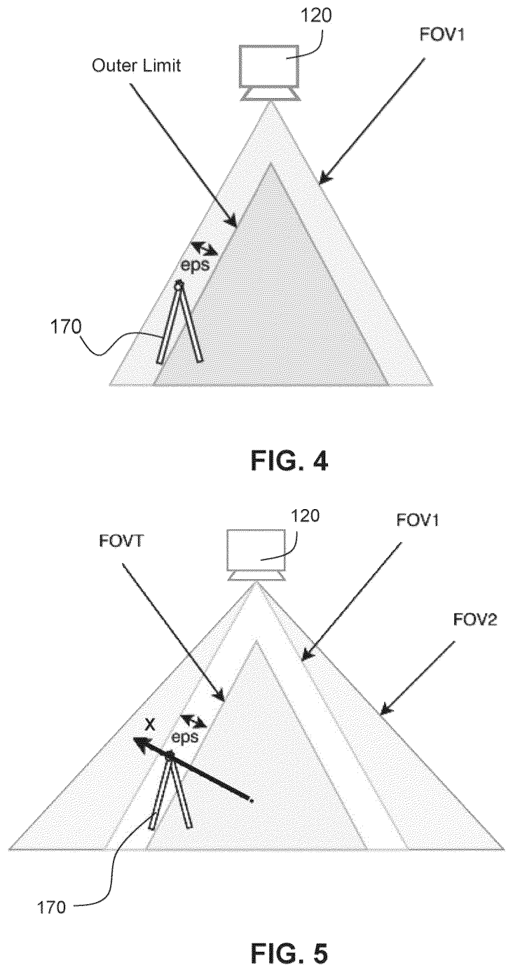

Several implementations of such an embodiment are shown in FIGS. 4-7.

In a particular embodiment (“Automatic unzoom”), shown in FIG. 5, the method includes commanding the viewing system to reduce the optical and/or digital zoom in real time, therefore enlarging the field of view, so that one or more instruments are made visible or constantly kept inside the new FOV called FOV2, in which:
FOV2⊃FOV1 (in the camera reference system);
the controlled pose of the slave device in the second viewing space FOV2 is at a distance greater than the threshold “eps”.

According to particular implementation options, provided by way of example:
“eps” is <⅕ of the size of the FOV and preferably 1/10;
“eps” is equivalent to 20% of the size of the slave device in the FOV.

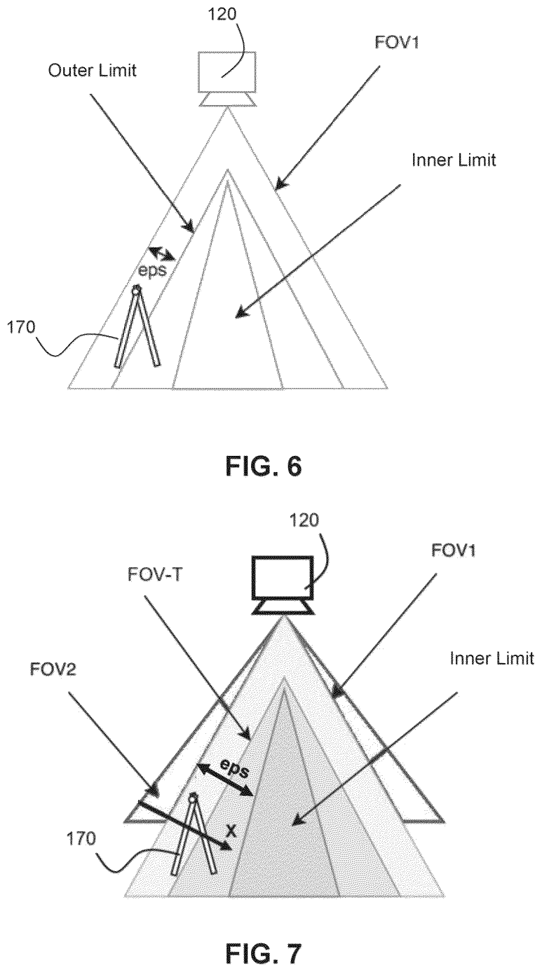

According to an implementation option, shown in FIG. 6, after widening the field of view (reaching the second viewing space FOV2), if the instrument returns inside the previous viewing space FOV1, the system is capable of controlling the zoom of the viewing system to return to a level which again identifies the starting FOV1. This is obtained by using a functional of distance which, if greater than a given threshold, leads to the transition from the second viewing space FOV2 to the first viewing space FOV1.

According to an implementation option, the first viewing space FOV1 is defined and stored at a considered start time, for example at the start of teleoperation of the robotic system.

In an embodiment, in the case of exit from the first viewing space FOV1, a plurality of “n” FOVs (FOV ₙ) is defined and progressively modified and suggested in real time, where the “n” FOVs are in a linear zoom relationship.

In an implementation option, there is a seamless solution between the initial FOV1 and the final FOV2, by dynamically and uniformly varying the zoom and focus parameters.

According to an implementation option, FOV2 is the smallest viewing space (in the camera space) capable of containing at least one surgical instrument of the slave device.

In an implementation option, the viewing space FOV must always contain at least two slave devices (i.e., two surgical instruments).

In an implementation option, the operator can, in a preoperative step or during the operation, define and/or save two different zoom values, and thus viewing space (FOV1,FOV2), within which the system can move autonomously when the instruments are exiting or entering with respect to the aforesaid first viewing space FOV1.

In particular, by bringing the surgical instruments to the limits or outside the first viewing space FOV1, the viewing system will autonomously switch to the second viewing space FOV2, keeping the instruments in the field of view.

While the surgical instruments are in FOV2, bringing the surgical instruments back towards the center of the FOV2 and within the volume covered by FOV1, the viewing system autonomously passes from FOV2 to FOV1.

In such an implementation option, FOV2⊃FOV1, i.e., FOV2 has a lower zoom than FOV1 and is capable of framing a larger portion of the surgical area within which the surgical instruments move.

In an implementation option, the aim is to keep the moving slave device in focus starting from the step of reaching the edge. In such a case, the focus is adjusted in the slave device, i.e., the slave device is returned to the “depth of field” region containing both the slave device and the initial work plane.

In an implementation option, the slave workspace contains the FOV1.

According to an implementation example, if the available zoom is not sufficient or the instrument reaches the limits of FOV2, the system exits teleoperation.

According to another implementation example, if the available zoom is not sufficient or the instrument reaches the limits of FOV2, the system does not exit teleoperation and the operator cannot exceed the limit of FOV3.

If the viewing system has reached the minimum zoom allowed or fails to return or keep at least one instrument in the FOV, the system exits teleoperation, or prevents exiting the FOV by means of appropriate constraints.

The teleoperated system applies a scaled Master-Slave movement in a possible range between 5-20 times. The scale factor can be used to change the aforesaid “eps” threshold, also taking into account the maximum speed of the slave device.

This mode is applicable to an analogue viewing system (“operating microscope”) provided with a camera, or to a digital viewing system (exoscope) connected to a screen. In both cases, an appropriate calculation unit analyzes the camera image and decides on commands to send to the viewing system. The aforesaid Control and Calculation Unit (CPU) can be located on the robot, on the viewing system, on an independent third element, on a representation system such as a screen.

In accordance with an embodiment (“Picture in Picture” or “PiP”), in which the viewing means further comprise a screen or monitor on which the digital images provided by the viewing system and processed by an appropriate calculation unit are represented, the viewing system in the same operating session simultaneously and continuously acquires in real time both the anatomical image of the magnified operating scenario FOV1 and an anatomical image of the operating scenario in an “unzoomed” or lower zoom mode having FOV2 and in which FOV2⊃FOV1.

In such a case, when a surgical instrument during a teleoperation session exits the FOV1 or is around or near the edges within an “eps” value, the anatomical image of the operating scenario in the “unzoomed” or lower zoom mode (FOV2) is shown to the user on said screen.

In an implementation option, it is shown in overlay on a portion of the screen where the FOV1 is also simultaneously represented.

When shown in overlay on a screen portion where the FOV1 is also simultaneously represented, the FOV2 is represented so as to cover a smaller screen section of the FOV1.

In particular, the aforesaid overlay is displayed, for example, in an area less than half the area of the screen and/or, in an area preferably located at one of the four corners of the screen.

In an implementation, the size and position of the insert are chosen using the golden ratio criterion, i.e., the insert has a side B, such that the remainder A is related so that A/B=(A+B)/A.

In an implementation option, in the moment of this transition, the FOV2 is represented with a higher FOV brightness, or FOV2 is represented with a colored or bright boundary.

In an implementation option, the second viewing space FOV2 is arranged on the screen in overlay from the side from which the instrument exited the first viewing space FOV1.

In an implementation option, FOV2 is shown on the whole screen and instead of the magnified image FOV1.

In an implementation option, the FOV2 occupies the entire screen while the FOV1 is displayed in overlay and a smaller size representation of the entire screen.

In an implementation option, the viewing means further comprise a secondary screen and the second viewing space FOV2 is represented on such a secondary screen.

In an implementation option, such a secondary screen is integral with the control console of the robotic system, in particular in a seating structure it is integral and extends from the armrest of the console.

In an implementation option, if the instrument also exits the field of view FOV2 of the anatomical image of the operating scenario in the “unzoomed” or lower zoom mode, the robotic system exits teleoperation.

In an implementation option, the viewing system and the robot are connected so that movements of the viewing system or modification of FOV parameters such as zoom are identified, monitored and recorded.

In an implementation option, the viewing system has a digital image acquisition sensor and is capable of acquiring both a portion of the “zoomed” sensor (FOV1) of the anatomical area and the entire sensor or a larger “unzoomed” (FOV2) portion thereof in real time and continuously.

In an implementation option, the viewing system is provided with two distinct and separate vision sensors or cameras, one defining FOV1 and one defining FOV2. In such a case, the point of view can be different.

In an implementation option, a FOV zoom change corresponds to a possible and automatic adjustment of exposure/brightness and/or focus.

In an implementation option, certain machine states and/or machine input and/or teleoperation system image analysis and/or teleoperation commands can trigger changes to viewing system parameters such as optical/digital zoom, focus, brightness, exposure, color filter.

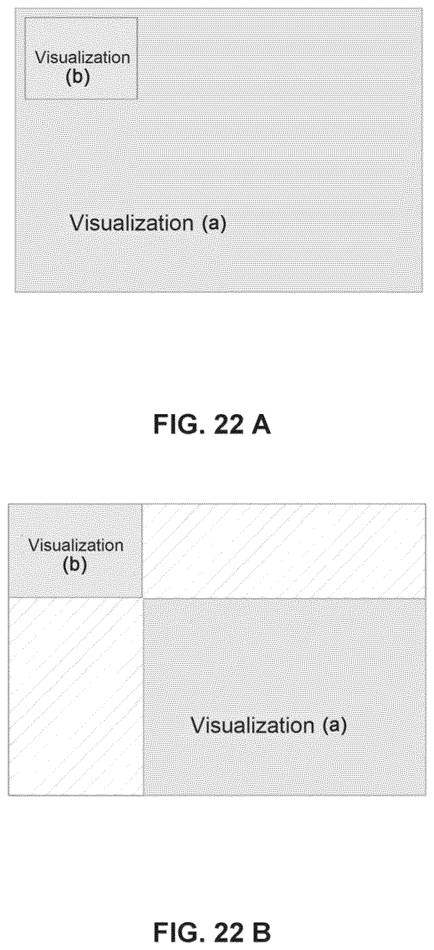

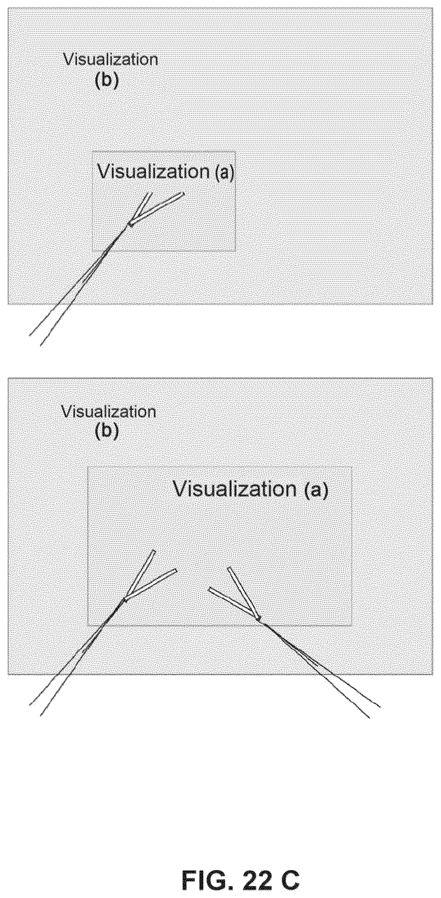

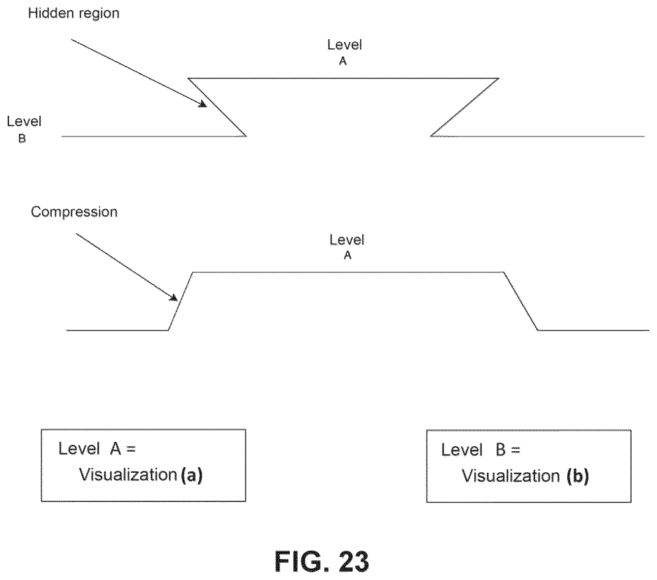

A further embodiment of the method is described below (shown in FIGS. 22A, 22B, 22C, 23).

In such an embodiment, the aforesaid second visualization and first visualization are defined herein, respectively, as “visualization b” (having a lower zoom value, thus with a larger field of view) and “visualization a” (having a higher zoom value, thus with a smaller field of view).

The following paradigms are provided for displaying “visualization a” and “visualization b” in a combined manner, according to the respective implementations of the embodiment described herein:
(i) not superimposed panels: “visualization a” and “visualization b” are not superimposed (as shown in FIG. 22A);
(ii) superimposed panels (Picture in Picture): “visualization a” covers the main area, while “visualization b” covers a smaller area (as shown in FIG. 22B);
(iii) “contextualized” visualization (with lens effect): “visualization a” covers a small area, “visualization b” covers the rest of the area (as shown in FIG. 22C) in which:
the region around the surgical instrument (or surgical instruments, if more than one) is displayed with a higher zoom (“visualization a”);
“visualization b” is distorted as if in “visualization a” it were applied by a lens (lens effect); this can occur by hiding an edge region between the two visualizations or by compressing an edge region between the two visualizations (as shown for example in FIG. 23).

According to an implementation option, the two-dimensional or three-dimensional nature of the image is taken into account. For example, the image of digital microscopes or exoscopes is three-dimensional, and therefore how the stereoscopic signal is treated must be considered.

According to an implementation example, this involves a two-dimensional “visualization b” and a three-dimensional “visualization a”, in the aforesaid cases (i) and (ii), while three-dimensional visualizations are considered for case (iii).

In accordance with an embodiment (“fusion”), a viewing system, a calculation unit and a screen on which the operator observes the operating field are available. The typical condition is to have the image taken by the viewing system on the screen with a given viewing space FOV.

In a condition (already previously considered) of the surgical instrument approaching the outer edge (or “Outer Limit”) of such an FOV, now called FOV1, the robotic system selects a second FOV2.

Such s second FOV2 is obtained by changing the parameters of the single camera system, or by means of a second camera system aligned with the first.

The “fusion” mode visually superimposes the results of the two viewing spaces FOV through image combination techniques so as to provide a higher resolution than that of the single view, i.e., a greater field depth.

Whatever the source of FOV1 and FOV2, the geometric relationship is known and in the simplest case the image provided by FOV1 is in the center of that of FOV2.

The distinction between this embodiment and the previous one (“Picture in Picture”) is that in this case the FOV1 is fused per-pixel to that of FOV2, enriching the information provided.

In a variant, during the switching from FOV1 to FOV2 the fusion system can express the transition dynamically seamlessly.

In the case of three-dimensional viewing systems, the fusion occurs by eye.

According to an embodiment of the method, the recognition of the surgical instruments, in the aforesaid digital images, occurs by means of the application of an appropriate deep neural network trained to identify, or the entire surgical instrument as an oriented rectangle, or a part thereof, or to separately identify all the parts thereof.

Based on the specificity level of the algorithm, a result can be obtained in the image space (pixel or normalized) or in the metric space if the camera is calibrated and the model of the identified objects is known.

The recognition performed with neural networks can be combined with position tracking techniques based on probabilistic models or even networks. This distinction between recognition and tracking is computationally useful since recognition is typically slower.

Once the polygon enclosing the surgical instrument part has been obtained, the distance thereof from the camera can be estimated and thus the distance from the FOV evaluated.

According to an embodiment, the viewing system is mounted on an articulated arm with sensors at the joints which are capable of identifying movements of the position thereof in space.

In an embodiment, the robotic system is capable of reconstructing the FOV and/or the intersection of FOV1 and FOV2 by calculating the possible movement of the viewing system and the parameters thereof such as zoom and distance.

In an embodiment, the robotic system, within the same operating session, stores, in a preparatory moment t1, the anatomical image of the operating scenario in an “unzoomed” mode or at a lower zoom than the teleoperation session later performed (at time t2) with higher zoom and FOV1 lower than FOV2.

When an instrument exits the FOV1 during a teleoperation session, the pre-recorded “static” anatomical image of the operating scenario in “unzoomed” or lower zoom mode is shown to the user in overlay on a screen portion where the magnified FOV is also represented or on a secondary screen. Such a representation only occurs if the viewing system has not been moved between t1 and t2.

In an embodiment, such representations of FOV1 and/or evaluations of the workspace limits thereof are inhibited if the system identifies the movement of the viewing system between t1 and t2 or after t2.

In an embodiment, the viewing system is mounted on an articulated arm with sensors at the joints or active controllable robotic arm, or the FOV is a magnified small subset of the actual field of view of a digital viewing system.

In such an embodiment, when the position commanded to the slave device is close by a given amount “eps” at the limits of the FOV, the viewing system moves to keep the instruments within a central FOV area.

The image acquisition device 120 can be adapted to acquire images according to two points of view and/or according to two different settings, thereby defining said viewing spaces FOV1 and FOV2.

Alternatively, or additionally, the image of a viewing space FOV2 can be acquired during a preparatory step, to be included in advance, and stored in a memory of the vision processing unit (e.g., in the case of a panoramic view of the surgical site 180).

In this case, the on-screen 130 visualization of the surgical device 170 when outside the first viewing space FOV1 and inside the second viewing space FOV2 can be processed by the system based on data from the motors of the slave surgical device 170 (thereby making a virtual on-screen 130 image of the slave surgical instrument when outside the first field of view 170).

With reference to what has been described above, and again to FIGS. 1-23, some embodiments will be presented below.

In accordance with an embodiment, the robotic system 100 comprises a pair of master control devices 110 which control a respective pair of slave robotic manipulators 171L, 171R, in which a slave surgical instrument 170 is connected to each robotic manipulator.

As shown for example in FIG. 1A, the image acquisition device 120, e.g., a camera 120, can be arranged between the robotic manipulators 171L, 171R of the pair, so that the field of view FOV1, FOV2 can include both the slave surgical instruments 170 of the pair, if necessary.

As shown for example still in FIG. 1A, the image acquisition device 120, for example a camera 120, can be mounted on an articulated arm 126, which for example is a robotic arm 126 (shown in FIG. 1B). Computing units 125 (or “Vision processing unit” 125) can be provided, arranged on the data connections 128, 129 between the image acquisition device 120 and the screen 130 as well as between the image acquisition device 120 and the master control device 110 as well as between the image acquisition device 120 and the slave device 170.

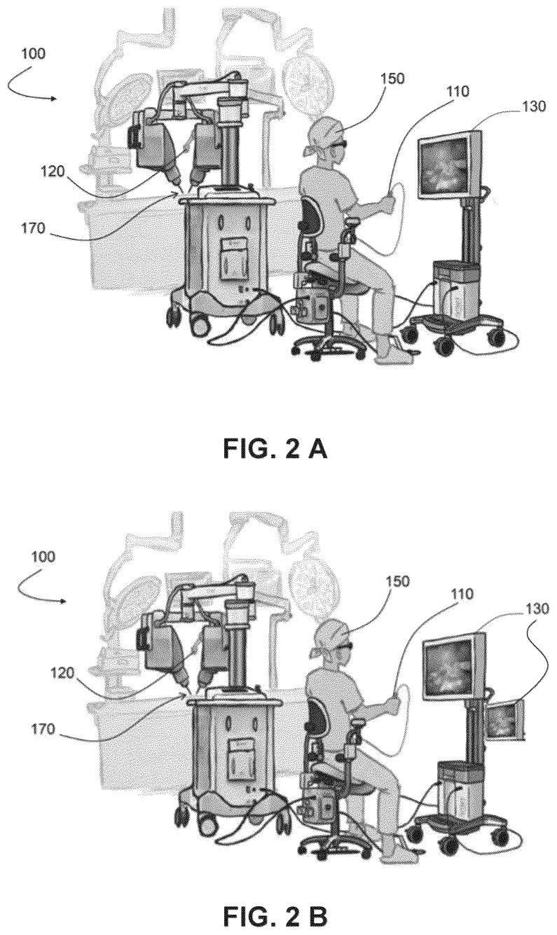

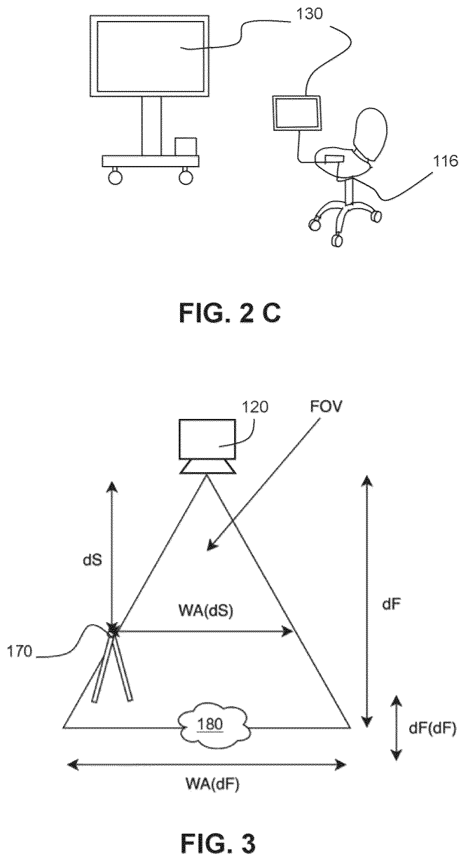

As shown for example in FIG. 2B, two screens 130 can be provided, which can be used to display two different superimposed viewing spaces FOV1, FOV2. As shown for example in FIG. 2C, a screen 130 can be mounted to a chair 116 of a master control console.

As shown for example in FIG. 4, a transition area (indicated in FIG. 4 with “eps”) can be defined, within the first viewing space FOV1, wherein, when the surgical instrument is located in said transition area eps, a transition to or from another viewing space FOV2 occurs.

As shown for example in FIG. 5, a portion FOVT of the viewing space FOV1 can thus be defined in which a viewing space transition does not occur, and a transition area eps, inside the boundaries of the first viewing space FOV1 in which the transition to the wide-field of view FOV2 occurs, when the surgical instrument 170 moves towards the outside of the field of view FOV1.

As shown for example in FIG. 6, the transition region eps can be provided when approaching the first narrow field of view FOV.

As shown for example in FIG. 7, the width of the transition region eps upon reentry in the narrow-field viewing space FOV1 can be different, larger in this example, from the transition area eps away from the narrow-field viewing space FOV1.

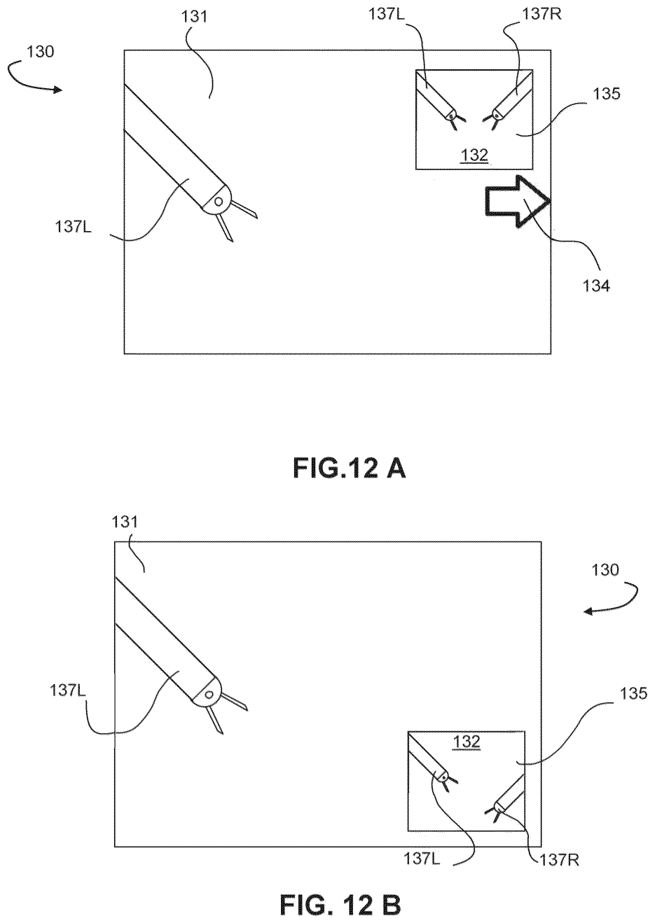

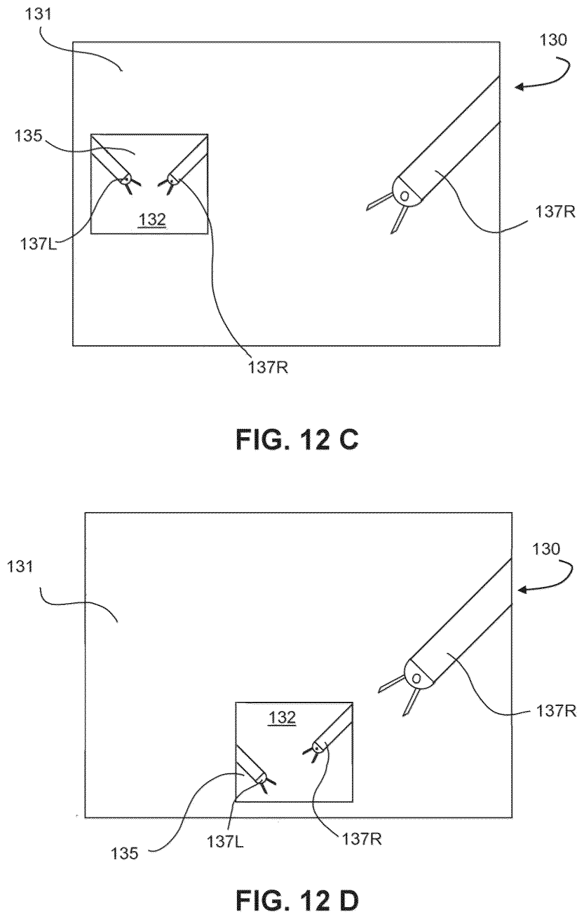

As shown for example in FIGS. 12A, 12B, 12C and 12D, an image 131 of the first viewing space FOV1 including an image 137L, 131R of a single slave surgical instrument 170 can be displayed on the screen or display 130, while an image 132 of the second viewing space FOV2 including images 137L, 137R of both slave surgical instruments of the pair is displayed as an image 135 in overlay. An indicator 134 for indicating the position of the other slave surgical instrument of the pair can be displayed in overlay on the image 131 of the first narrow-field viewing space FOV1.

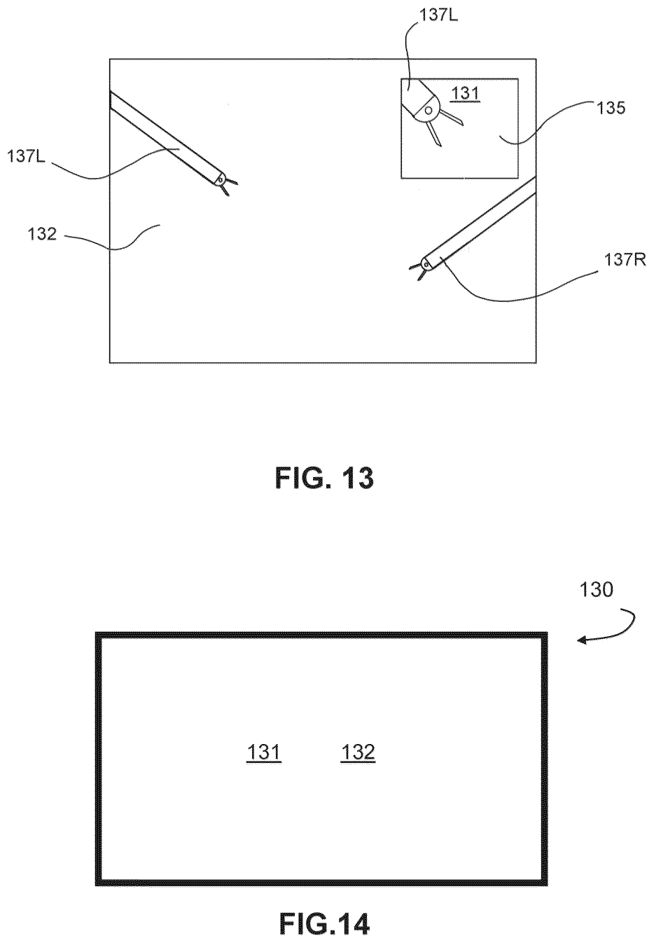

The image 135 displayed in overlay can be the image 131 of the first viewing space FOV1 with higher zoom, as shown for example in FIG. 13, or vice versa, can be the image 132 of the second wide-field viewing space FOV.

As shown for example in FIG. 14, an image which is the digital fusion of the images 131 and 132 related to the respective viewing spaces FOV1, FOV2 can be shown on the screen 130.

The image acquisition device 120 can comprise a single digital sensor 121 capable of acquiring a wide viewing space FOV2 and an image processing unit 125 generating a digital magnification (zoom) of a portion of said wide viewing space, thereby defining the viewing space FOV1, as shown for example in FIG. 16A. This avoids the necessity to provide optical systems to be moved.

As shown for example in FIG. 16B, the digital sensor 121 can be associated with an optical unit 123 (which can comprise one or more lenses) which determine the magnifications (zoom) and an automatic movement system of the optical unit 123 can be provided.

As shown for example in FIG. 16C, two coordinated sensors 121, 122 can be provided to generate a three-dimensional (3D) image, for example by means of the image processing unit 125.

As shown for example in FIG. 16D, the viewing spaces FOV1 and FOV2 can be acquired by different sensors 121, 122 with different points of view. The narrow and wide viewing spaces FOV1, FOV2 can both be included in the slave workspace 175 of the slave device 175, for example the space of the joints of the manipulators 171L, 171R, or the slave workspace 175 can be included entirely in the wide field of view FOV2.

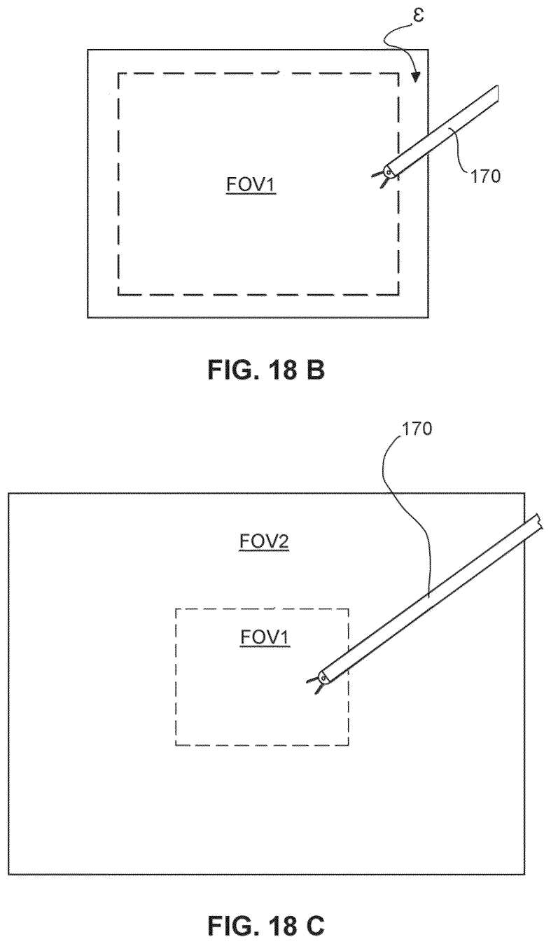

As shown for example in FIGS. 18A, 18B and 18C, when the slave surgical instrument 170 is moved along a direction X away from the viewing space FOV1, and is located inside an area ε within the boundaries of the viewing space FOV1, the system can automatically switch to the wide second viewing space FOV2. The detection of the slave surgical instrument 170 within the spatial tolerance area ε can occur by means of digital visual identification (Computer Vision) to determine the transition to the second viewing space FOV2 and/or the detection of the slave surgical instrument 170 within the spatial tolerance area E can occur by means of control over the input commands provided by the master control device 110 (i.e., by the operator 150).

For example, after detecting and discriminating a command imparted to the master control device 110 such as to drive the slave surgical instrument 170 outside the field of view FOV1, the system initiates digital visual identification (Computer vision) to manage the transition times towards a wide second viewing space FOV2.

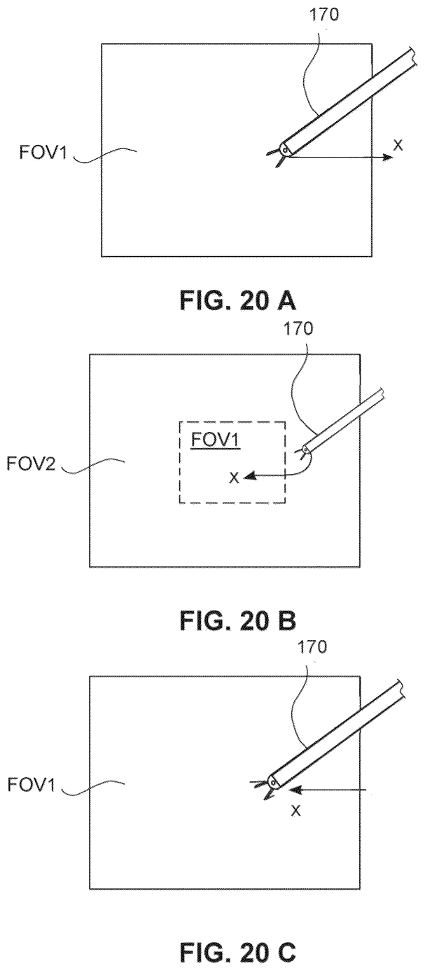

As shown for example in FIGS. 20A, 20B and 20C, if the slave surgical instrument 170 enters the narrow first field of view FOV1, the system can be configured to automatically switch to the narrow first field of view FOV1.

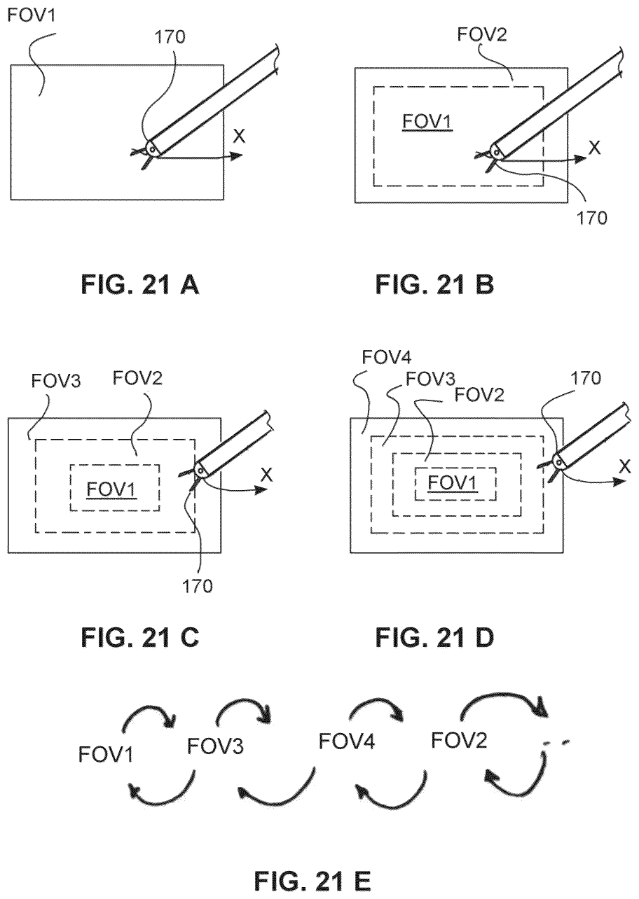

A plurality of gradually wider fields of view can be provided (as shown for example in FIGS. 21A to 21E).

In accordance with an embodiment, the zoom, i.e., the instantaneous magnification level, continuously decreases or increases within a predetermined range, and the image processing unit 125 can be configured to optimize the zoom level to keep the slave surgical instrument 170 always within the current viewing space (e.g., FOV1, FOV2, FOV3, and/or FOV4, as shown for example in FIGS. 21A-21E and 22A-22C).

As can be seen, the objects of the present invention as previously indicated are fully achieved by the method and system disclosed above by virtue of the features described above in detail.

Those skilled in the art may make changes and adaptations to the embodiments of the method and system described above or can replace elements with others which are functionally equivalent in order to meet contingent needs without departing from the scope of the following claims. Each of the features described above as belonging to a possible embodiment can be implemented irrespective of the other embodiments described.

LIST OF REFERENCE SIGNS

| | |
|---|---|
| 100 | Robotic system |
| 110 | Master control device |
| 116 | Operator chair |
| 120 | Image acquisition device (means) |
| 121 | Image acquisition sensor or chip |
| 122 | Image acquisition sensor or chip |
| 123 | Optical unit |
| 125 | Video processing unit, or image processing unit |
| 126 | Positioning and support arm of the image |
| | acquisition device |
| 128 | Connection of the image acquisition device to the |
| | robotic system |
| 129 | Connection to the image acquisition device screen |
| 130 | Screen (display means) |
| 131 | Image of the first viewing space FOV1 |
| 132 | Image of the second viewing space FOV2 |
| 134 | Indicator |
| 135 | Element in overlay, or overlay |
| 137, 137L, | Slave surgical instrument image |
| 137R | |
| 150 | Operator or surgeon |
| 170 | Slave device, or slave surgical instrument |
| 171L, 171R | Slave robotic manipulator |
| 175 | Slave device workspace, or “slave workspace” |
| 180 | Surgical site |
| FOV | Viewing space |
| FOV1 | First viewing space |
| FOV2 | Second viewing space |
| FOV3, FOV4 | Other viewing space |
| FOVT | Portion of stability viewing space |
| eps | Threshold or transition region |
| X | Slave surgical instrument movement |
| ε | Spatial tolerance area |

1. A method for controlling a robotic system for medical or surgical teleoperation, comprising at least one surgical instrument, adapted to operate in teleoperation, and viewing means configured to display to an operator images and/or videos of at least one viewing space associated with a teleoperation area in which the surgical instrument operates, wherein the method comprises:
determining whether a position of the surgical instrument is within an allowed space correlated to a first viewing space, wherein the first viewing space is used as a current viewing space for the teleoperation, with respect to which a current visualization is provided to the operator;
automatically providing a second visualization, by the viewing means, when in said determining step, a determination is made that said position of the surgical instrument is not within said allowed space correlated to the first viewing space,
wherein said second visualization defines a second viewing space having a greater surface or a greater field of view than said first viewing space and containing, or partially containing, said first viewing space;
and wherein said second visualization comprises:
a combination and/or superimposition of said second viewing space and said first viewing space, and/or
a switching from said first viewing space to said second viewing space by controlling optical and/or digital parameters of said viewing means without mechanical movements.

2. A method according to claim 1, wherein said step of automatically providing a second visualization comprises displaying to the operator a combination and/or superimposition of said second viewing space and said first viewing space, in images and/or videos, by display means included in said viewing means.

3. A method according to claim 1, wherein said step of automatically providing a second visualization comprises displaying to the operator the second viewing space through a switching from said first viewing space to said second viewing space, by controlling, without mechanical movements, optical and/or digital parameters of image acquisition means comprised in said viewing means,
wherein said optical parameters comprise, electronic control of zoom and/or exposure and/or focus and/or depth of focus, and said digital parameters comprise, digital scale factor and/or magnification and/or region of interest and/or digital zoom or other digital image processing.

4. A method according to claim 1, wherein said step of determining a position of the surgical instrument comprises determining a current position of the surgical instrument with respect to the allowed space correlated to the first viewing space, and/or presence of the surgical instrument in the allowed space correlated to the viewing space, and
said step of automatically providing a second visualization comprises automatically providing a second visualization when the current determined position of the surgical instrument is on a boundary of and/or outside said allowed space correlated to the first viewing space.

5. A method according to claim 4, wherein said current position of the surgical instrument is detected based on digital images, provided in real time by image acquisition means included in the viewing means.

6. A method according to claim 1, wherein said robotic system comprises at least one master device adapted to be moved by an operator, and further comprises said at least one surgical instrument adapted to be controlled by the master device,
wherein said step of determining a position of the surgical instrument comprises determining a position of the surgical instrument with respect to the first viewing space, as imposed by the master device, and
said step of automatically providing a second visualization comprises automatically providing a second visualization when the determined controlled position of the surgical instrument is on a boundary of outside and/or inside said allowed space correlated to the first viewing space.

7. A method according to claim 4, wherein said step of automatically providing a second visualization comprises ensuring that the surgical instrument is displayed in said second visualization provided to the operator, during movement of the surgical instrument as controlled by the master device, even when the surgical instrument moves outside the allowed space correlated to the first viewing space, during teleoperation or preparation for teleoperation.

8. A method according to claim 1, wherein said second viewing space is a physical viewing space, detectable by the image acquisition means and displayable by the display means in addition to the first viewing space, or wherein said second viewing space is a virtual viewing space, extractable or extrapolatable from digital images and/or videos previously detected and/or displayable by the viewing means in addition to the first viewing space.

9. A method according to claim 3, comprising the further steps of:
acquiring, by the image acquisition means, both a first digital video image of a magnified operating scenario, defined in said first viewing space, and a second digital video image of the enlarged operating scenario, defined in said second viewing space, which includes the first viewing space and in which the surgical instrument is displayed, said second viewing space being wider and/or at a lower zoom than the first viewing space; and
wherein said step of automatically providing a second visualization, when the determined position of the surgical instrument is on a boundary of or outside the allowed space correlated to the first viewing space, comprises displaying both the first viewing space and the second viewing space.

10. A method according to claim 9, wherein said step of displaying both the first viewing space and the second viewing space comprises displaying the second viewing space, in which the surgical instrument is visible, in overlay on a screen portion, covering a part of the first viewing space, wherein the screen portion in which the second viewing space is shown in overlay comprises an area located at one of four corners of the screen, and/or a side box, arranged on a side of the first viewing space from which the surgical instrument exited from the space correlated to the first viewing space, or
wherein said step of displaying both the first viewing space and the second viewing space comprises displaying the second viewing space, in which the surgical instrument is visible, in full screen, and displaying the first viewing space in overlay on a screen portion, covering a part of the second viewing space in which the surgical instrument is not present, or
wherein the method further comprises highlighting the second viewing space by increased brightness or with a colored or bright edging, and/or a sound effect at each transition between the first viewing space and the second viewing space and vice versa, or at a moment of transition between the first visualization and the second visualization.

11-13. (canceled)

14. A method according to claim 3, comprising the further steps of:
acquiring, by the image acquisition means, both a first digital video image of a magnified operating scenario, defined in said first viewing space, and a second digital video image of the enlarged operating scenario, defined in said second viewing space, which includes the first viewing space and in which the surgical instrument is displayed, said second viewing space being wider and/or at a lower zoom than the first viewing space; and
wherein said step of automatically providing a second visualization, when the determined position of the surgical instrument is on the boundary of or outside the allowed space correlated to the first viewing space, comprises:
processing both the first acquired digital video image and the second acquired digital video image, by image processing techniques, to perform a pixel-by-pixel merging of the two digital images and obtain, as a second visualization provided to the operator, a combined digital image of the operating scenario, based on said image merging.

15. A method according to claim 1, wherein the step of providing the second visualization comprises switching from a first digital video image of the operating scenario, taken at a first zoom value, to a second digital image of the operating scenario, taken by a second zoom value lower than the first zoom value, and by a second viewing space which is wider than the first viewing space,
wherein said switching step is performed by the viewing means by controlling optical and/or digital parameters, and free of controlling movement parameters of the viewing means,
wherein, if the surgical instrument returns within the allowed space associated with the first viewing space, the first visualization is restored.

16. A method according to claim 15, wherein a plurality of gradually larger second viewing spaces and a respective plurality of gradually lower second zoom values, is provided, and
wherein said step of automatically providing a second visualization, when the determined position of the surgical instrument is on the boundary of or outside an allowed space correlated to a second current viewing space comprises displaying the next wider second viewing space having a respective lower second zoom value, to display the surgical instrument.

17. (canceled)

18. A method according to claim 1, wherein the robotic system remains in teleoperation until the surgical instrument is displayed in said first and/or second visualization,
wherein if the surgical instrument reaches boundaries of the second field of view, and the second field of view cannot be further enlarged, the robotic system exits the teleoperation state, and/or
wherein, if the surgical instrument reaches the boundaries of the second field of view, and the second field of view cannot be further enlarged, the robotic system remains in a limited teleoperation state, in which movement of the surgical instrument is allowed only if the surgical instrument is located within an allowed space correlated to the second viewing space and the movement of the surgical instrument, if allowed, is confined within said second viewing space.

19. (canceled)

20. A method according to claim 1, wherein said allowed space correlated to the viewing space corresponds to the viewing space, and/or
wherein said allowed space correlated to the viewing space comprises a subset of the viewing space, corresponding to the viewing space from which an internal surrounding, extending with a spatial tolerance within the boundaries of the viewing space, is removed,
and/or wherein said viewing space is defined by:
a field of view of the viewing means, and/or
a predefined subset of the field of view of the viewing means, and/or
a field-of-view workspace, comprising a geometric volume, in a reference coordinate system of the robotic system, associated with said field of view, and/or
geometric limits of the field of view, comprising a boundary surface of said view workspace, in the reference coordinate system of the robotic system.

21. (canceled)

22. A method according to claim 1, wherein said step of determining a current position of the surgical instrument with respect to the allowed space correlated to the viewing space comprises:
mapping said allowed space correlated to the viewing space in a corresponding slave field-of-view workspace, in a slave reference coordinate system associated with the slave device;
determining the current position of the surgical instrument in terms of respective position coordinates in said slave reference coordinate system;
determining the current position of the surgical instrument with respect to the allowed space correlated to the viewing space based on a comparison between said position coordinates and said slave field-of-view workspace, in the slave reference coordinate system.

23. A method according to claim 1, wherein said step of determining a position of the surgical instrument with respect to the allowed space correlated to the viewing space comprises:
calculating and/or determining a position of an actual point belonging to the surgical instrument or a position of a virtual point integral with the surgical instrument, and/or
calculating and/or determining a position of a virtual control point of the slave device, and/or
determining a position of at least one tip of the surgical instrument, and/or
determining a position of at least one link of a hinged wrist included in the surgical instrument, and/or
determining a position of a distal portion of a positioning shaft proximate the hinged wrist of the surgical instrument.

24. A robotic system for medical or surgical teleoperation, comprising:
at least one surgical instrument, adapted to operate in teleoperation;
viewing means configured to display images and/or videos to the operator of at least one viewing space associated with a teleoperation area in which the surgical instrument operates,
a control unit configured to control the surgical instrument, wherein the control unit is further configured to:
determine whether a position of the surgical instrument is within an allowed space correlated to a first viewing space, wherein the first viewing space is used as the current viewing space for the teleoperation, with respect to which a current visualization is provided to the operator;
automatically provide a second visualization, by the viewing means, when in said determining step, a determination is made that said position of the surgical instrument is not within said allowed space correlated to the first viewing space,
wherein said second visualization defines a second viewing space having a greater surface or a greater field of view than said first viewing space and containing, or partially containing, said first viewing space;
and wherein said second visualization comprises:
a combination and/or superimposition of said second viewing space and said first viewing space, and/or
a switching, by controlling optical and/or digital parameters of said viewing means without mechanical movements, from said first viewing space to said second viewing space.

25. (canceled)

26. A robotic system according to claim 24, further comprising:
at least one master device adapted to be moved by an operator;
at least one slave device comprising said surgical instrument adapted to be controlled by the master device;
and wherein the viewing means comprise:
image acquisition means configured to acquire a digital image or video associated with said first viewing space and/or second viewing space;
display means, configured to display to the operator said first viewing space or said second viewing space, and/or a combination and/or superimposition of said second viewing space and said first viewing space.

27. A robotic system according to claim 26, wherein the image acquisition means comprise two or more image sensors or cameras, having a same viewpoint or two different respective viewpoints, configured to perform said actions of acquiring a first digital video image and a second digital video image simultaneously, continuously and in real time,
wherein said two or more image sensors or cameras comprise two sensors or cameras adapted to provide two-dimensional viewing spaces, or two sensors or cameras adapted to provide one three-dimensional viewing space, or four sensors or cameras adapted to provide two three-dimensional viewing spaces, or
wherein said two image sensors comprise two cameras having said first viewing space and said second viewing space as a viewing space, respectively, aligned and/or coaxial with each other.

28. (canceled)

29. A robotic system according to claim 26, wherein the image acquisition means comprise a single image sensor or camera, configured to alternately perform said actions of acquiring a first digital video image and a second digital video image, wherein said image sensor or camera is configured to detect said first digital video image associated with the first viewing space by adjusting zoom to a first zoom value, and detect said second digital video image associated with the second viewing space by adjusting the zoom to a second zoom value;
wherein said image sensor or camera is configured to perform, upon switching from the first digital video image to the second digital image, or vice versa, in addition to switching from the first zoom value to the second zoom value, or vice versa, an automatic controlled variation and/or automatic adjustment of other optical parameters, including exposure/brightness and/or focusing.

30-47. (canceled)
